(19) United States
(12) Patent Application Publication
(10) Pub. No.: US 2025/0195388 A1
(43) Pub. Date: Jun. 19, 2025

(54) HAIR SHAPING COMPOSITIONS AND METHODS FOR SHAPING HAIR

(71) Applicant: L'OREAL, Paris (FR)

(72) Inventors: Cho-Cho KHINE, Scotch Plains, NJ (US); Ronak RUGHANI, Edison, NY (US)

(73) Assignee: L'OREAL, Paris (FR)

(21) Appl. No.: 18/539,954

(22) Filed: Dec. 14, 2023

(51) Int. Cl. A61K 8/73; A61Q 5/06

(52) U.S. Cl. CPC A61K 8/738; A61Q 5/06; A61K 2800/5426; A61K 2800/80

(57) ABSTRACT

The present disclosure is drawn to hair shaping compositions and methods for shaping hair, comprising: (i) applying a hair shaping composition according to the instant disclosure to the hair; (ii) physically manipulating the hair upon which the hair shaping composition is applied into a desired shape; and (iii) without removing the hair shaping composition from the hair, drying the hair, and optionally heating the hair. The hair shaping composition includes: (a) citric acid, a salt thereof, or a combination thereof; (b) one or more cyclodextrins, derivatives thereof, or a combination thereof; (c) one or more water soluble solvents; (d) one or more cationic guar derivatives; and (e) water. Hair treated with the hair shaping compositions has a desired shape and also exhibits improved fiber alignment, frizz control, and smoothness.

### FIELD OF THE DISCLOSURE

The present disclosure is drawn to hair shaping compositions and to methods for shaping the hair. Hair treated with the compositions exhibits improved fiber alignment, frizz control, and smoothness.

### BACKGROUND

Many consumers use cosmetic and care compositions to enhance the appearance of hair, e.g., by changing the color, style, or shape of the hair and/or by imparting various cosmetic properties to hair, such as shine and conditioning. Hair can become dry or damaged for various reasons, e.g., weather exposure, poor nutrition, mechanical treatments (e.g., brushing hair), styling treatments using chemicals, dying, heat, nutrition, etc. Even cleansing products can remove hair's natural oils causing dryness, which can lead to a dull appearance, split ends, and frizz.

Chemical treatments for hair include bleaching and coloring treatments to change the color the hair. Chemical treatments also include processes to permanently change the shape and structure of the hair, for example by perming, waving, relaxing or straightening the hair. These chemical treatments change the look of hair by changing its physical structure, which inevitably causes a certain degree of damage to the hair. Environmental factors, such as salt water, sunlight, and heat, are also known to damage hair. Damaged hair is characterized by unnatural changes to the protein structure of the individual hair strands or shafts.

The popularity and usage of oils for hair treatments has increased due to their effectiveness and simplicity. Commonly used oils include olive oil, mineral oil, avocado oil, apricot kernel oil, rice bran oil, and coconut oil. However, these treatments can leave the hair feeling greasy. In addition, the effects are not usually seen after more than several hours (e.g., 8 hours) of treatment and several treatments are usually required, making it time consuming and labor intensive.

Damage to hair results in split ends, dryness, hair that is easily broken, and hair that becomes “frizzy” and unmanageable. Because the visible portion of hair is dead, it has no ability to regenerate itself. There are numerous over the counter and salon treatments that purport to repair damaged hair. These include conditioners, hot oil treatments, hydrolyzed proteins, vitamin formulations, and exotic fruit, leaf, or root extracts. These treatments, however, provide only limited improvement to the hair. Therefore, hair treatment technologies that can shape, curl, straighten, relax, or style the hair without chemically damaging the hair are desired.

There is still a need for providing improved hair curling, waving, shaping, straightening, smoothing, and manageability, for example, improved hair alignment, reduced unwanted volume (especially reduced frizz), and increased shine.

Further, the formulation of environmentally friendly cosmetic products, which are designed and developed considering environmental issues, is becoming a major goal in an effort to meet global challenges. It is therefore essential to propose more sustainable compositions, preparation processes and ingredients to address these environmental concerns. In this context, it is important to develop new cosmetic formulations with a better carbon footprint, particularly by promoting the use of renewable raw materials and/or cosmetic compositions with a good index of naturalness and/or materials of natural origin and, more particularly, materials of plant origin while reducing the use of compounds of petrochemical origin.

### SUMMARY OF THE DISCLOSURE

The instant disclosure is drawn to hair shaping compositions and methods for shaping and smoothing hair, for example curling hair. Hair treated with the compositions has a desired shape in addition to exhibiting improved fiber alignment, reduced frizz, and smoothness. Further, the hair is soft, shiny, conditioned, and has a healthy appearance. The compositions include a combination of citric acid, or salt thereof, and a cyclodextrin, or derivative thereof. The citric acid, or salt thereof, and the cyclodextrin, or derivative thereof, are separately combined with each other and the cyclodextrin or derivative is dissolved in the citric acid. Subsequently, the combination is incorporated into the hair shaping composition.

The hair shaping compositions typically include:
(a) citric acid, a salt thereof, or a combination thereof;
(b) one or more cyclodextrins, derivatives thereof, or a combination thereof;
(c) one or more water soluble solvents;
(d) one or more cationic guar derivatives; and
(e) water.

Citric acid and its salts provide a variety of benefits to hair. For example, it acts as an antioxidant, removes build-up and debris from the hair, and improves blood circulation in the scalp, which in turn nourishes hair follicles and promotes growth. Its acidic pH is also useful for balancing the pH of the scalp, as many hair care products make are alkaline. The citric acid in the hair shaping composition also interacts with cyclodextrin in a unique manner and improves film formation on the hair, which is further potentiated with heat.

Cyclodextrins are a family of cyclic oligosaccharides, comprised of a macrocyclic ring of glucose subunits joined by α-1,4 glycosidic bonds. Cyclodextrins are produced from starch by enzymatic conversion. Typical cyclodextrins contain a number of glucose monomers ranging from six to eight units in a ring, creating a cone shape, for example, α (alpha)-cyclodextrin gas 6 glucose subunits, β (beta)-cyclodextrin has 7 glucose subunits, and γ (gamma)-cyclodextrin has 8 glucose subunits. Nonlimiting examples of cyclodextrins for use in the compositions of the present disclosure include α-cyclodextrin, β-cyclodextrin, γ-cyclodextrin, methyl-α-cyclodextrin, methyl-β-cyclodextrin, methyl-γ-cyclodextrin, and mixtures thereof.

The weight ratio of the citric acid, salts thereof, or combination of (a) to the cyclodextrin or derivatives of (b) is typically from about 4:1 to about 1:1 ((a):(b)) and/or the mole ratio of the citric acid, salts thereof, or combination of (a) to the cyclodextrin or derivatives of (b) is typically from about 20:1 to about 5:1 ((a):(b)).

The citric acid, salts thereof, or combination of (a) and the cyclodextrin, derivatives thereof, or combination thereof of (b) are typically combined with one another before being combined with the other ingredient of the hair shaping composition. For example, the cyclodextrin and/or derivatives thereof (cyclodextrin component) are preferably dissolved or solubilized in the citric acid, salt thereof, or combination thereof (citric acid component) to form a solubilized combination of citric acid and cyclodextrin. The combination of cyclodextrin component and citric acid component can be heated to facilitate or hasten the dissolution of the cyclodextrin component in the citric acid component. When the cyclodextrin component is individually dissolved in the citric acid component prior to combination with other ingredients of the hair shaping compositions, the cyclodextrin/citric acid component provides unique properties to the hair shaping composition that differ from the properties that result from combining the cyclodextrin component and the citric acid component with the other ingredients of the composition without first dissolving the cyclodextrin component in the citric acid component.

Nonlimiting examples of water soluble solvents include C₂-C₆ mono-alcohols (e.g., ethanol, isopropyl alcohol, etc.), polyols (polyhydric alcohols), glycerin, glycols (e.g., ethylene glycol, propylene glycol, butylene glycol, pentylene glycol, caprylyl glycol, etc.), and mixtures thereof. Glycerin, also known as glycerol or glycerine, is a simple triol (C₃H₈O₃). It is a colorless, odorless, and viscous liquid that acts as a natural humectant by attracting and moisture from its surroundings.

Cationic guar derivatives include cationically modified guar and hydroxypropyl guar and alkyl guar, in particular, cationic hydroxylpropyl guar, as well as salts thereof. A typical cationic functional group in these cation guar derivatives is trimethylamino (2-hydroxyl) propyl, with a counter ion such as a halide (e.g., chloride, fluoride, bromide, iodide), sulfate, or methylsulfate. Nonlimiting exemplary cationic guar derivatives include cationically modified alkylated carboxylated polymers such as methyl and ethyl carboxymethyl guar and cationic hydroxypropyl guar, in particular, cationic hydroxypropyl guar.

The hair shaping compositions are aqueous compositions containing significant amounts water. In addition to water, the compositions optionally include one or more water soluble solvents other than glycerine. Nonlimiting examples of water-soluble solvents include C₂-C₆ mono-alcohols (e.g., ethanol, isopropyl alcohol, etc.), polyols (polyhydric alcohols), glycols (e.g., ethylene glycol, propylene glycol, butylene glycol, pentylene glycol, caprylyl glycol, etc.), and mixtures thereof. In various embodiments, at least one of the one or more water soluble solvents is glycerin, a glycol (e.g., ethylene glycol, propylene glycol, butylene glycol, pentylene glycol, caprylyl glycol, etc.) or mixtures thereof.

The pH of the hair shaping compositions is typically acidic, for example, from about 3 to about 6.5, preferably from about 3 to about 6, more preferably from about 3 to about 5.

The hair shaping composition may optionally include one or more miscellaneous ingredients, for example, one or more preservatives, fragrances, pH adjusters, salts, chelating agents, buffers, antioxidants, flavonoids, vitamins, amino acids, botanical extracts, UV filtering agents, peptides, proteins, protein hydrolysates, and/or isolates, fillers (e.g., organic and/or inorganic fillers such as talc, calcium carbonate, silica, particular materials, etc.), emollients, composition colorants, or mixtures thereof.

Methods for shaping hair employing the hair shaping compositions include:
(i) applying a hair shaping composition of the instant disclosure to hair;
(ii) physically manipulating the hair upon which the hair shaping composition is applied to provide the hair with a desired shape;
(iii) without removing the shaping composition from the hair, drying the hair; and
(iv) optionally, heating the hair, wherein the heating of the hair may optionally be carried out after a pause time.

The hair shaping composition is applied to the hair and the hair is physically manipulated into a desired shape. For example, the hair can be rolled onto rollers to curl the hair, or the hair can be combed, brushed, or pulled, for example, with fingers and hands, to curl or straighten the hair. In addition, a round brush can be used to roll the hair while drying the hair to form curls or waves in the hair. The hair shaping composition can be messaged or spread throughout the hair with an individual hands or a comb or brush can be used to spread the hair shaping composition uniformly throughout the hair.

The hair is physically manipulated to form a desired shape. For example, to curl the hair, the hair can be wound onto curlers. Once the hair is manipulated to form a desired shape, the hair is dried while being held in the desired shape. For instance, wet or damp hair upon which the hair shaping composition is applied can be wrapped around curlers and dried while wrapped around the curlers. The hair may naturally dry under ambient conditions, for example, at a temperature of about 25° C. Once dry, the hair can be removed from the curlers. The hair can optionally be treated with a thermal treatment (a heat treatment) to dry and/or to shape the hair. A heat treatment is not required but in various embodiments, a heat treatment is preferred.

The heat treated may be preceded by a pause time. A “pause time” is a period of time between application of the hair shaping composition to the hair and treating the hair with a heat treatment. For example, the hair shaping composition may be applied to the hair, after which a pause time is employed before treating the hair with a heat treatment. The pause time begins after the hair shaping composition has been applied to the hair before the hair becomes dry. After the pause time, the hair is treated with a heat treatment, for example, with a blow drying or a hot iron such as a curling iron or a flat iron at a temperature of about 140° C. to about 220° C. The heat treatment can be used to aid in drying the hair and contribute to shaping of the hair or the heat treatment can be applied to the hair after the hair is dried, e.g., after a pause time. The pause time can vary but is typically from about 1 minute to about 2 hours. In a preferred embodiment, the hair is treated with a hair shaping composition and after a pause time of about 1 minute to about 1 hour, the hair is treated with a heat treatment, wherein the heat treatment is preferably treatment with a hot iron, such as a curling iron or a flat iron. The hair may dry naturally during the pause time or can be dried with a blow dryer.

### BRIEF DESCRIPTION OF THE DRAWINGS

Implementation of the present technology is described, by way of example only, with reference to the attached figures, wherein:

FIG. 1 shows curl retention of hair swatches treated with an inventive composition or a control after 5 minutes, 30 minutes, 24 hour, 24 hours, and 96 hours in a humidity chamber of 80% RH at 23° C.

FIG. 2 compares curl length of hair swatches treated with an inventive composition or a control after 5 minutes, 30 minutes, 24 hour, 24 hours, and 96 hours in a humidity chamber of 80% RH at 23° C.

FIG. 3 shows curl retention of hair swatches treated with an inventive composition or a control and a curling iron after 5 minutes, 30 minutes, 24 hour, 24 hours, and 96 hours in a humidity chamber of 80% RH at 23° C.

FIG. 4 compares curl length of hair swatches treated with an inventive composition or a control and a curling iron after 5 minutes, 30 minutes, 24 hour, 24 hours, and 96 hours in a humidity chamber of 80% RH at 23° C.

It should be understood that the various aspects are not limited to the arrangements and instrumentality shown in the drawings.

### DETAILED DESCRIPTION OF THE DISCLOSURE

The instant disclosure is drawn to hair shaping compositions and to methods for shaping hair. Hair treated according to the disclosure has a desired shape and exhibits improved fiber alignment, reduced frizz, and smoothness. The hair shaping effects can be enhanced with heat, for example, with a hot iron, but heating is not necessary. Regardless of whether heat is employed, the shape of the hair is long-lasting and durable, even under high humidity conditions.

The hair shaping composition typically includes:
(a) about 1 to about 8 wt. % of citric acid, a salt thereof, or a combination thereof;
(b) about 0.5 to about 5 wt. % of one or more cyclodextrins, derivatives thereof, or a combination thereof;
   wherein a combined total amount of (a) and (b) is about 2 to about 12 wt. %;
(c) about 0.1 to about 5 wt. % of glycerin;
(d) about 0.1 to about 8 wt. % of one or more cationic guar derivatives; and
(e) about 70 to about 96 wt. % of water;
   wherein all percentages by weight are based on a total weight of wherein all percentages by weight are based on a total weight of the composition.

The hair shaping composition is used in methods for shaping hair. The manner in which the hair is shaped is not limited. Nonetheless, in various embodiments, the hair shaping composition is used to curl the hair. In various embodiments, the hair shaping composition is used to straighten the hair. Methods of shaping the hair include:
(i) applying a hair shaping composition according to the instant disclosure to the hair;
(ii) physically manipulating the hair upon which the hair shaping composition is applied into a desired shape; and
(iii) without removing the shaping composition from the hair, drying the hair, and optionally treating the hair with a heat treatment, wherein the heat treatment is optionally preceded by a pause time.

### (a) Citric Acid and Salts Thereof

The total amount of citric acid, salts thereof, or combination thereof will vary. Nonetheless, in various embodiments, the hair shaping composition includes about 1 to about 8 wt. % of citric acid, salts thereof, or a combination thereof, based on a total weight of the composition. In further embodiments, the hair shaping composition includes about 1 to about 6 wt. %, about 1 to about 4 wt. %, about 1 to about 3 wt. %, about 2 to about 8 wt. %, about 2 to about 6 wt. % about 2 to about 5 wt. %, about 2 to about 4 wt. %, about 2 to about 3 wt. %, about 2.5 to about 8 wt. %, about 2.5 to about 6 wt. %, about 2.5 to about 5 wt. %, about 2.5 to about 4 wt. %, about 2.5 to about 3 wt. %, or about 1, about 1.5, about 2, about 2.5, about 3, about 3.5, about 4, about 4.5, or about 5 wt. % of citric acid, salts thereof, or a combination thereof, based on a total weight of the composition.

### (b) Cyclodextrin and Derivatives

The hair shaping compositions include one or more cyclodextrins or derivatives thereof. As used herein, the term “cyclodextrins” includes slats of carboxylic acid, whether or not expressly stated. Cyclodextrins are a family of cyclic oligosaccharides consisting of a macrocyclic ring of glucose subunits joined by α-1,4 glycosidic bonds. The cyclodextrins that can be used include those of the formula:

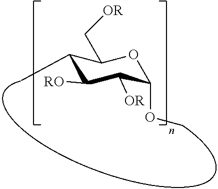

wherein:
R is chosen from H, CH₃, or a hydroxypropyl group, and
n ranges from 6-8.

For example, in embodiments where R═H, the cyclodextrin may be α-cyclodextrin (n=6), β-cyclodextrin (n=7), or γ-cyclodextrin (n=8). By way of example, α-cyclodextrin sold by the company WACKER under the name CAVAMAX W6 PHARMA, β-cyclodextrin sold by the company WACKER under the name CAVAMAX W7 PHARMA, or γ-cyclodextrin sold by the company WACKER under the name CAVAMAX W8 PHARMA can be used.

In other embodiments where R═CH₃, the cyclodextrin may be a methyl-cyclodextrin, such as methyl-α-cyclodextrin (n=6), methyl-β-cyclodextrin (n=7), or methyl-γ-cyclodextrin (n=8). For example, the methyl-β-cyclodextrin sold by the company WACKER under the name CAVASOL W7 may be chosen.

In various embodiments, the at least one cyclodextrin may comprise a mixture of cyclodextrins and/or derivatives thereof. For example, the at least one cyclodexctrin may be a mixture of α-cyclodextrin, β-cyclodextrin, and/or γ-cyclodextrin. In another embodiment, the at least one cyclodextrin includes β-cyclodextrin. In yet a further embodiment, the cyclodextrin is only β-cyclodextrin, and no other cyclodextrins or derivatives thereof are present in the composition.

In one embodiment, the hair shaping compositions according to the present disclosure includes β-cyclodextrin in an amount ranging from about 0.1% to about 10%, such as from 0.2% to about 8%, from about 0.3% to about 7%, from about 0.4% to about 6%, from about 1% to about 10%, from about 1% to about 8%, from about 1% to about 5%, from about 1% to about 3% by weight, relative to the total weight of the hair shaping composition.

The total amount of cyclodextrin in the hair shaping compositions will vary. Nonetheless, in various embodiments, the hair shaping composition includes about 0.5 to about 5 wt. % of cyclodextrin, based on a total weight of the composition. In further embodiments, the hair shaping composition includes 0.5 to about 4 wt. %, about 0.5 to about 3 wt. %, about 0.5 to about 2 wt. %, about 1 to about 5 wt. %, about 1 to about 4 wt. %, about 1 to about 3 wt. %, about 1 to about 2 wt. %, about 1.5 to about 5 wt. %, about 1.5 to about 4 wt. %, about 1.5 to about 3 wt. %, about 1.5 to about 2 wt. %, about 0.5 wt. %, about 1 wt. %, about 1.25 wt. %, about 1.5 wt. %, about 2 wt. %, about 2.5 wt. %, about 3 wt. %, about 3.5 wt. %, about 4 wt. %, about 4.5 wt. %, or about 5 wt. %, based on a total weight of the composition.

### Combination of Citric Acid/Salts and Cyclodextrin

The total combined amount of the citric acid, salts thereof, or combination of (a) and the cyclodextrin or derivatives of (b) will vary. Nonetheless, in various embodiments the total combined amount of the citric acid, salts thereof, or combination of (a) and the cyclodextrin and derivatives of (b) is about 1 to about 12 wt. %, based on a total weight of the hair shaping composition. In further embodiments, the total combined amount of the citric acid, salts thereof, or combination of (a) and the cyclodextrin and derivatives of (b) is about 1 to about 10 wt. %, about 1 to about 8 wt. %, about 1 to about 6 wt. %, about 1 to about 5 wt. %, about 2 to about 12 wt. %, about 2 to about 10 wt. %, about 2 to about 8 wt. %, about 2 to about 6 wt. %, about 2 to about 5 wt. %, about 3 to about 12 wt. %, about 3 to about 10 wt. %, about 3 to about 8 wt. %, about 3 to about 6 wt. %, about 3 to about 5 wt. %, or about 1 wt. %, 2 wt. %, 3 wt. %, 4 wt. %, 5 wt. %, 6 wt. %, 7 wt. %, or 8 wt. %, based on a total weight of the hair shaping composition.

The weight ratio of the citric acid, salts thereof, or combination of (a) to the cyclodextrin or derivatives of (b) will vary. Nonetheless, in various embodiments, the citric acid, salts thereof, or combination of (a) and the cyclodextrin or derivatives of (b) are in a weight ratio of about of about 8:1 to about 1:2 ((a):(b)). In further embodiments, the citric acid, salts thereof, or combination of (a) and the cyclodextrin or derivatives of (b) are in a weight ratio of 6:1 to about 1:2, about 5:2 to about 1:2, about 4:1 to about 1:2, about 3:1 to about 1:2, about 2:1 to about 1:2, about 8:1 to about 1:1, about 6:1 to about 1:1, about 5:1 to about 1:1, about 4:1 to about 1:1, about 3:1 to about 1:1, about 2:1 to about 1:1, about 1.2:1, about 1.3:1, about 1.4:1, about 1.5:1, about 1.6:1, or about 1.8:1 ((a):(b)).

The mole ratio of the citric acid, salts thereof, or combination of (a) to the cyclodextrin or derivatives of (b) will vary. Nonetheless, in various embodiments, the citric acid, salts thereof, or combination of (a) and the cyclodextrin or derivatives of (b) are in a mole ratio of about 20:1 to about 3:1. In further embodiments, the citric acid, salts thereof, or combination of (a) and the cyclodextrin or derivatives of (b) are in a mole ratio of about 18:1 to about 3:1, about 15:1 to about 3:1, about 20:1 to about 5:1, about 18:1 to about 5:1, about 15:1 to about 5:1, about 20:1 to about 8:1, about 18:1 to about 8:1, about 15:1 to about 8:1, about 20:1 to about 10:1, about 18:1 to about 10:1, about 15:1 to about 10:1, about 14:1, about 13:1, about 12:1, or about 11:1.

In various embodiments, the citric acid, salts thereof, or combination of (a) and the cyclodextrin of (b) are combined with one another before being added into the hair shaping compositions of the instant disclosure. For example, the cyclodextrin is preferably solubilized in the citric acid to form a solubilized combination of citric acid and cyclodextrin. The combination can be heated to facilitate or hasten the dissolution of the cyclodextrin. The solubility of cyclodextrin in water is not always ideal. Therefore, combining the cyclodextrin with the citric acid and dissolving the cyclodextrin in citric acid before adding combining the combination with other components of the hair shaping composition can be beneficial.

### (c) Water Soluble Solvent

The term “water soluble organic solvent” is interchangeable with the terms “water soluble solvent” and “water-miscible solvent” and means a compound that is liquid at 25° C. and at atmospheric pressure (760 mmHg), and it has a solubility of at least 50% in water under these conditions. In some cases, the water-soluble solvents have a solubility of at least 60%, 70%, 80%, or 90%. Non-limiting examples of water-soluble solvents include, for example, organic solvents selected from glycerin, alcohols (for example C₁₋₈, or C₁₋₄ alcohols), polyols (polyhydric alcohols), glycols, and a mixture thereof.

Nonlimiting examples of water-soluble organic solvents. Non-limiting examples of water-soluble organic solvents include, for example, organic solvents selected from alcohols (for example, C₁₋₁₀, C₁₋₈, or C₁₋₄ alcohols), polyols (polyhydric alcohols), glycols, and a mixture thereof. Nonlimiting examples of monoalcohols and polyols include ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, and propane diol.

Further non-limiting examples of water soluble organic solvents include alkanediols (polyhydric alcohols) such as 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

Polyhydric alcohols are useful. Examples of polyhydric alcohols include ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof.

In a preferred embodiment, the hair straightening composition includes one or more glycols selected from propylene glycol, butylene glycol, pentylene glycol, hexylene glycol, caprylyl glycol, dipropylene glycol, and mixtures thereof.

In a preferred embodiment, the hair straightening composition includes glycerin.

The amount of water soluble solvents in the hair shaping compositions will vary. Nonetheless, the total amount of one or more water soluble solvents may be from about 0.1 to about 20 wt. %, based on the total weight of the hair straightening composition. In further embodiments, the hair shaping composition include from about 0.1 to about 15 wt. %, about 0.1 to about 10 wt. %, about 0.1 to about 5 wt. %, about 1 to about 20 wt. %, about 1 to about 15 wt. %, about 1 to about 10 wt. %, about 1 to about 5 wt. %, based on the total weight of the hair shaping composition.

In further embodiments, the hair shaping composition includes from about 0.1 to about 5 wt. % of one or more water soluble solvents, based on the total weight of the hair shaping composition. In further embodiments, the hair shaping composition includes from about 0.1 to about 4 wt. %, about 0.1 to about 3 wt. %, about 0.1 to about 2 wt. %, about 0.2 to about 5 wt. %, about 0.2 to about 4 wt. %, about 0.2 to about 3 wt. %, about 0.2 to about 2 wt. %, about 0.5 to about 5 wt. %, about 0.5 to about 4 wt. %, about 0.5 to about 3 wt. %, about 0.5 to about 2 wt. %, about 0.8 to about 5 wt. %, about 0.8 to about 4 wt. %, about 0.5 to about 3 wt. %, about 0.8 to about 2 wt. % of one or more water soluble solvents, based on the total weight of the hair shaping composition.

In even further embodiments, the hair shaping composition includes from about 0.1 to about 5 wt. % of glycerin, based on the total weight of the hair shaping composition. In additional embodiments, the hair shaping composition includes from about 0.1 to about 4 wt. %, about 0.1 to about 3 wt. %, about 0.1 to about 2 wt. %, about 0.2 to about 5 wt. %, about 0.2 to about 4 wt. %, about 0.2 to about 3 wt. %, about 0.2 to about 2 wt. %, about 0.5 to about 5 wt. %, about 0.5 to about 4 wt. %, about 0.5 to about 3 wt. %, about 0.5 to about 2 wt. %, about 0.8 to about 5 wt. %, about 0.8 to about 4 wt. %, about 0.5 to about 3 wt. %, about 0.8 to about 2 wt. % of glycerin, based on the total weight of the hair shaping composition.

### (d) Cationic Guar Derivatives

Cationic guar derivatives include cationically substituted galactomannan (guar) gum derivatives. The guar molecule itself is a straight chain mannan branched at regular intervals with single membered galactose units on alternative mannose units. The mannose units are linked to each other by means of β (1-4) glycosidic linkages. The galactose branching arises by way of an α (1-6) linkage. Cationic derivatives of the guar gums are obtained by reaction between the hydroxyl groups of the polygalactomannan and reactive quaternary ammonium compounds. The degree of substitution of the cationic groups onto the guar structure must be sufficient to provide a desired cationic charge density. Suitable quaternary ammonium compounds for use in forming the cationic guar polymers include those conforming to the general Formula (XII):

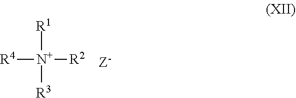

wherein where R1, R2 and R3 are methyl or ethyl groups;
R4 is either an epoxyalkyl group of the general Formula (XIII):

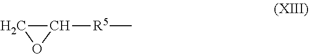

or R4 is a halohydrin group of the general Formula (XIV):

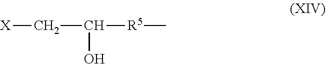

wherein R5 is a C1 to C3 alkylene; X is chlorine or bromine, and Z is an anion such as Cl—, Br—, I— or HSO4-.

Cationic guar polymers (cationic derivatives of guar gum) formed from the reagents described above are represented by the general Formula (XV):

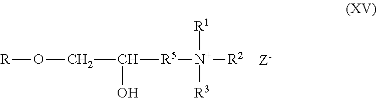

wherein R is guar gum. Preferably, the cationic guar polymer is guar hydroxypropyltrimethylammonium chloride, which can be more specifically represented by the general Formula (XVI):

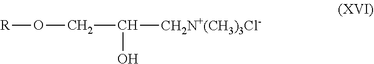

Suitable cationic guar derivatives have cationic charge densities of at least about 1.25 meq/gm, preferably at least about 1.5 meq/gm, more preferably at least about 1.7 meq/gm, even more preferably at least about 1.8 meq/gm, still more preferably at least about 2.0 meq/gm, and even more preferably at least about 2.3 meq/gm, but also preferably less than about 7 meq/gm, more preferably less than about 5 meq/gm, still more preferably less than about 4.5 meq/gm. The “cationic charge density” of a cationic guar derivative, as that term is used herein, refers to the ratio of the number of positive charges on a monomeric unit of which the polymer is comprised to the molecular weight of said monomeric unit. The cationic charge density multiplied by the polymer molecular weight determines the number of positively charged sites on a given polymer chain.

Any anionic counterions may be use in association with the cationic guars so long as the cationic guars remain soluble in water, in the hair shaping composition, or in a coacervate phase of the hair shaping composition, and so long as the counterions are physically and chemically compatible with the essential components of the hair shaping composition or do not otherwise unduly impair product performance, stability or aesthetics. Nonlimiting examples of such counterions include: halides (e.g., chloride, fluoride, bromide, iodide), sulfate, methylsulfate, and mixtures thereof.

Nonlimiting examples of cationic guar derivatives include cationic hydroxyethyl guar, cationic hydroxypropyl guar, cationic hydroxybutyl guar, cationic carboxymethyl guar, cationic carboxylpropyl guar, cationic carboxybutyl guar, carboxymethylhydroxypropyl guar, guar hydroxypropyltrimonium chloride, and hydroxypropyl guar hydroxypropyltrimonium chloride.

The amount of the cationic guar derivative in the hair shaping compositions will vary. Nonetheless, in various embodiments, the hair shaping composition includes from about 0.1 to about 8 wt. % of the cationic guar derivative, based on the total weight of the hair shaping composition. In further embodiments, the hair shaping composition includes from about 0.1 to about 5 wt. %, about 0.1 to about 4 wt. %, about 0.1 to about 3 wt. %, about 0.1 to about 2 wt. %, about 0.2 to about 5 wt. %, about 0.2 to about 4 wt. %, about 0.2 to about 3 wt. %, about 0.2 to about 2 wt. %, about 0.5 to about 5 wt. %, about 0.5 to about 4 wt. %, about 0.5 to about 3 wt. %, about 0.5 to about 2 wt. %, about 0.8 to about 5 wt. %, about 0.8 to about 4 wt. %, about 0.5 to about 3 wt. %, about 0.8 to about 2 wt. % of the cationic guar derivative, based on the total weight of the hair shaping composition.

### (e) Water

The total amount of water in the hair shaping compositions will vary. Nonetheless, in various embodiments, the hair shaping compositions include about 70 to about 96 wt. % of water, based on the total weight of the compositions. In further embodiments, the hair shaping composition includes about 75 to about 96 wt. %, about 80 to about 96 wt. %, about 85 to about 96 wt. %, about 90 to about 96 wt. %, about 70 to about 95 wt. %, about 75 to about 95 wt. %, about 80 to about 95 wt. %, about 85 to about 95 wt. %, about 90 to about 95 wt. %, based on the total weight of the hair shaping composition.

### Miscellaneous Ingredients

The compositions optionally include or exclude (or are essentially free from) one or more miscellaneous ingredients. Miscellaneous ingredients are ingredients that are compatible with the compositions and do not disrupt or materially affect the basic and novel properties of the compositions. Nonlimiting examples of ingredients include preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, etc. In various embodiments, the miscellaneous ingredients are chosen from preservatives, fragrances, pH adjusters, salts, chelating agents, buffers, composition colorants, and mixtures thereof. In the context of the instant disclosure, a “composition colorant” is a compound that colors the composition but does not have an appreciable coloring effect on hair. In other words, the composition colorant is included to provide a coloring to the composition for aesthetic appeal but is not intended to impart coloring properties to hair. Styling gels, for example, can be found in a variety of different colors (e.g., light blue, light pink, etc.) yet application of the styling gel to hair does not visibly change the color of the hair.

The total amount of the one or more miscellaneous ingredients in the compositions, if present, will vary. Nonetheless, in various embodiments, the compositions include about 0.1 to about 15 wt. % of the one or more miscellaneous ingredients, based on the total weight of the compositions. In further embodiments, the compositions include about 0.1 to about 12 wt. %, about 0.1 to about 10 wt. %, about 0.1 to about 5 wt. %, about 0.5 to about 15 wt. %, about 0.5 to about 12 wt. %, about 0.5 to about 10 wt. %, about 0.5 to about 8 wt. %, about 0.5 to about 5 wt. %, about 1 to about 15 wt. %, about 1 to about 12 wt. %, about 1 to about 10 wt. %, about 1 to about 8 wt. %, about 1 to about 5 wt. %, about 2 to about 15 wt. %, about 2 to about 12 wt. %, about 2 to about 10 wt. %, about 2 to about 8 wt. %, or about 2 to about 5 wt. %, based on the total weight of the compositions.

pH

The pH of the composition will vary. Nonetheless, the compositions typically have a pH of less than 7 (an acidic pH). For example, the pH can be from about 2.5 to about 6.5, about 2.5 to about 6, about 2.5 to about 5.5, about 2.5 to about 5, about 2.5 to about 4.5, about 2.5 to about 4, about 3 to about 6.5, about 3 to about 6, about 3 to about 5.5, about 3 to about 5, about 3 to about 4.5, or about 3 to about 4.

### Methods

As explained throughout the instant disclosure, the hair shaping compositions are particularly useful in methods for shaping hair, preferably human hair, in particular human hair of the head. Treatment with the compositions improves fiber alignment, reduces frizz, and impart smoothness to the hair. The methods typically comprise applying a hair shaping composition of the instant disclosure to the hair. The hair is preferably wet or damp, but the composition can also be applied to dry hair. Application of the compositions to the hair will moisten the hair because the compositions are aqueous (contain a large portion of water).

The hair shaping composition is spread throughout the hair, preferably spread substantially uniformly throughout the hair, as a leave-in product, i.e., the hair shaping composition is not typically rinsed from the hair prior to shaping and/or drying the hair. The hair upon which the hair shaping composition is applied can be physically straightened or physically manipulated with a shaping action. For example, a comb or brush can be used to incorporate the hair shaping composition throughout the hair while simultaneously shaping the hair, i.e., pulling the hair in a manner that straightens the hair. Similarly, the hair can be physically manipulated with one's hands to spread the hair shaping composition throughout the hair and the hands can also be used to pull or lay the hair flat to straighten the hair.

While the hair is being brushed or physically manipulated with a shaping action, the hair can dry naturally under ambient conditions. Alternatively, a blow dryer or hair dryer can be used to expedite drying of the hair. For example, the hair can be combed or brushed while being blow dried to simultaneously straighten and dry the hair. The blow dryer can provide heat to the hair and expedite the shaping and drying process. In various embodiments, the hair is heated to a temperature of about 120° C. to about 250° C., preferably 140° C. to about 250° C., more preferably about 150° C. to about 200° C. In various embodiments, after the hair is dry or is mostly dry, the hair can be treated with a thermal treatment (treated with heat). For example, the hair can be treated with a hot iron, in particular, a flat iron. Typically, the hot iron is passed over the hair at least once, at least twice, at least three times, or more. The hot iron is preferably at a temperature of about 120° C. to about 250° C., preferably 140° C. to about 250° C., more preferably about 150° C. to about 200° C.

In various embodiments, the method includes a pause time between application of the hair shaping composition to the hair and a thermal treatment. The pause time begins immediately after application of the hair shaping composition to the hair, while the hair still wet. The hair may dry naturally during the pause time, or the hair can be subjected to a drying process to expedite drying of the hair, for example, the hair can be dried with a blow dryer, provided the blow dryer does not heat the hair according to a subsequent thermal treatment (heat treatment). After a pause time, a thermal treatment (heat treatment) is applied to the hair. For example, the thermal treatment preferably heats the hair to a temperature of about 120° C. to about 250° C., preferably 140° C. to about 250° C., more preferably about 150° C. to about 200° C. A blow dryer can provide the heat treatment provided the blow dryer heats the hair to the expressed temperatures. In a preferred embodiment, the hot iron is used for the heat treatment, of example, a curling iron or a flat iron, wherein the hot iron heats the hair to a temperature of about 120° C. to about 250° C., preferably 140° C. to about 250° C., more preferably about 150° C. to about 200° C.

When a pause is employed between application of the hair shaping composition and a thermal treatment, the pause time will vary but is typically from about 1 minutes to about 2 hours. In further embodiments, the pause time is from about 1 minute to about 1 hour, about 1 minute to about 30 minutes, about 1 minute to about 20 minutes, about 2 minutes to about 2 hours, about 2 minutes to about 1 hour, about 2 minutes to about 30 minutes, about 2 minutes to about 20 minutes, about 5 minutes to about 2 hours, about 5 minutes to about 1 hour, about 5 minutes to about 30 minutes, about 5 minutes to about 20 minutes, about 15 minutes to about 2 hours, about 15 minutes to about 1 hour, or about 15 minutes to about 30 minutes.

### Exclusions

All components that are positively set forth in the instant disclosure may be negatively excluded from the claims, e.g., a claimed composition may be “free,” “essentially free” (or “substantially free”) of one or more components that are positively set forth in the instant disclosure.

In various embodiments, the hair shaping composition is free or essentially free from anionic surfactants.

In various embodiments, the hair shaping composition is free or essentially free from polymers, copolymers, and crosspolymers formed with acrylate or methacrylate monomers, e.g, free or essentially free from polyacrylic acid and polyacrylate polymers and crosspolymers.

In various embodiments, the hair shaping composition is free or essentially free from N-alkyl-2-mercaptoacetamide. In further embodiments, the hair shaping composition is free or essentially free from all mercaptoacetamides.

In various embodiments, the hair shaping composition is free or essentially free from ethylene carbonate. In further embodiments, the hair shaping composition is free or essentially free from free from linear carbonates, e.g., dimethyl carbonate, diethyl carbonate, etc. In various embodiments, the composition is free or essentially free from cyclic lactones (e.g., valerolactone, caprolactone, pantolactone, meadowlactone, etc.), free or essentially free from heterocyclic molecules (e.g., 2-oxazolidinone, 2-imidazolidinone, etc.) free or essentially free from sulfones (dimethyl sulfone, 2,3,4,5-tetrahydrothiophene-1,1-dioxide), and/or free or essentially free from ureas (e.g., urea, ethylene urea, etc.). In yet further embodiments, the hair shaping composition is free or essentially free from carbonates. For example, the hair shaping composition may be free or essentially free from cyclic carbonates, preferably cyclic carbonates selected from propylene carbonate, dipropylene carbonate, butylene carbonate, 2,3-butylene carbonate, 2,3-pentylene carbonate, pentylene carbonate, ethylene carbonate, glycerol carbonate, or a mixture thereof.

In various embodiments, the hair shaping compositions are free or essentially free from polysaccharides other than the cyclodextrin and/or derivatives thereof and the cationic guar derivatives.

In various embodiments, the hair shaping composition is free or essentially free from silicones, including amino-functionalized silicones such as amodimethicone.

In further embodiments, the hair shaping composition is free or essentially free from monosaccharides and disaccharides. For example, the composition is free or essentially free from ribose, arabinose, glucose, fructose, xylose, sucrose, and/or methyl glucoside.

In various embodiments, the hair shaping composition is free or essentially free from formaldehyde, derivatives of formaldehyde, formalin, and compounds that produce formaldehyde upon heating.

In further embodiments, the hair shaping composition is free or essentially free from thioglycolic acid, thiolactic acid, or salts thereof.

### EMBODIMENTS

In various embodiments, the hair shaping composition comprises or consists of:
(a) about 1 to about 8 wt. %, preferably about 1 to about 6 wt. %, more preferably about 2 to about 5 wt. % of citric acid, salt thereof, or combination thereof;
(b) about 0.5 to about 5 wt. %, preferably about 1 to about 5 wt. %, more preferably about 1 to about 3 wt. % of one or more cyclodextrins, derivatives thereof, or a combination thereof;
   wherein a combined total amount of the citric acid, salt thereof, or combination thereof of (a) and the one or more cyclodextrins, derivatives thereof, or combination thereof of (b) is about 2 to about 12 wt. %, preferably about 2 to about 8, more preferably about 3 to about 6 wt. %; and
   wherein preferably (a) and (b) are individually combined with each other to form a composition in which (b) is dissolved in (a) before subsequently being combined with additional components of the hair-treatment composition;
(c) about 0.1 to about 5 wt. %, preferably about 0.1 to about 3 wt. %, more preferably about 0.5 to about 2 wt. % of one or more water soluble solvents, wherein preferably the one or more water soluble solvents are selected from C₂-C₆ mono-alcohols (e.g., ethanol, isopropyl alcohol, etc.), polyols (polyhydric alcohols, such as glycerin), glycols (e.g., ethylene glycol, propylene glycol, butylene glycol, pentylene glycol, caprylyl glycol, etc.), and mixtures thereof, wherein more preferably at least one of the one or more water soluble solvents is glycerin;
(d) about 0.1 to about 8 wt. %, preferably about 0.2 to about 5 wt. %, more preferably about 0.5 to about 4 wt. % of one or more cationic guar derivatives, wherein the one or more cationic guar derivatives are preferably selected from cationic hydroxyethyl guar, cationic hydroxypropyl guar, cationic hydroxybutyl guar, cationic carboxymethyl guar, cationic carboxylpropyl guar, cationic carboxybutyl guar, carboxymethylhydroxypropyl guar, guar hydroxypropyltrimonium chloride, and hydroxypropyl guar hydroxypropyltrimonium chloride, preferably wherein the one or more cationic guar derivatives is guar hydroxypropyltrimonium chloride, hydroxypropyl guar hydroxypropyltrimonium chloride, or a mixture thereof;
(e) about 70 to about 96 wt. %, preferably about 80 to about 95 wt. %, more preferably about 85 to about 95 wt. % of water;
   wherein all percentages by weight are based on a total weight of the composition.
(f) optionally, about 0.1 to about 15 wt. %, preferably about 0.5 to about 10 wt. %, more preferably about 0.5 to about 5 wt. % of one or more water soluble solvents, preferably wherein the one or more water soluble solvents are selected from C₂-C₆ mono-alcohols, polyols (polyhydric alcohols), glycols, or a mixture thereof;
(g) optionally, about 0.01 to about 10 wt. %, preferably about 0.1 to about 8 wt. %, more preferably about 1 to about 5 wt. % of one or more miscellaneous ingredients, preferably selected from preservatives, fragrances, pH adjusters, salts, chelating agents, buffers, antioxidants, flavonoids, vitamins, amino acids, botanical extracts, UV filtering agents, peptides, proteins, protein hydrolysates, and/or isolates, fillers (e.g., organic and/or inorganic fillers such as talc, calcium carbonate, silica, particular materials, etc.), emollients, composition colorants, or a mixture thereof;
   wherein all percentages by weight are based on a total weight of the composition.

The pH of the composition can vary. Nonetheless, in various embodiments, a pH less than 7 (an acidic pH) is desirable. For example, the pH can be from about 3 to about 6.5, about 3 to about 6, about 3 to about 5.5, about 3 to about 5, about 3 to about 4.5, about 3.5 to about 6.5, about 3.5 to about 6, about 3.5 to about 5.5, about 3.5 to about 5, or about 3.5 to about 4.5.

In various embodiments, the hair shaping composition comprises, consists essentially of, or consists of:
(a) about 1 to about 8 wt. %, preferably about 1 to about 6 wt. %, more preferably about 2 to about 5 wt. % of citric acid, salt thereof, or combination thereof;
(b) about 0.5 to about 5 wt. %, preferably about 1 to about 5 wt. %, more preferably about 1 to about 3 wt. % of one or more cyclodextrins, derivatives thereof, or a combination thereof;
   wherein a combined total amount of the citric acid, salt thereof, or combination thereof of (a) and the one or more cyclodextrins, derivatives thereof, or combination thereof of (b) is about 2 to about 12 wt. %, preferably about 2 to about 8, more preferably about 3 to about 6 wt. %;
   wherein preferably (a) and (b) are individually combined with each other to form a composition in which (b) is dissolved in (a) before subsequently being combined with additional components of the hair-treatment composition;
(c) about 0.1 to about 5 wt. %, preferably about 0.1 to about 3 wt. %, more preferably about 0.5 to about 2 wt. % of one or more water soluble solvents, wherein preferably the one or more water soluble solvents are selected from C₂-C₆ mono-alcohols (e.g., ethanol, isopropyl alcohol, etc.), polyols (polyhydric alcohols, such as glycerin), glycols (e.g., ethylene glycol, propylene glycol, butylene glycol, pentylene glycol, caprylyl glycol, etc.), and mixtures thereof, wherein more preferably at least one of the one or more water soluble solvents is glycerin;
(d) about 0.1 to about 8 wt. %, preferably about 0.2 to about 5 wt. %, more preferably about 0.5 to about 4 wt. % of guar hydroxypropyltrimonium chloride, hydroxypropyl guar hydroxypropyltrimonium chloride, or a mixture thereof;
(e) about 70 to about 96 wt. %, preferably about 80 to about 95 wt. %, more preferably about 85 to about 95 wt. % of water;
   wherein all percentages by weight are based on a total weight of the composition;
(f) optionally, about 0.1 to about 15 wt. %, preferably about 0.5 to about 10 wt. %, more preferably about 0.5 to about 5 wt. % of one or more water soluble solvents, preferably wherein the one or more water soluble solvents are selected from C₂-C₆ mono-alcohols, polyols (polyhydric alcohols), glycols, or a mixture thereof; and
(g) optionally, about 0.01 to about 10 wt. %, preferably about 0.1 to about 8 wt. %, more preferably about 1 to about 5 wt. % of one or more miscellaneous ingredients, preferably selected from preservatives, fragrances, pH adjusters, salts, chelating agents, buffers, antioxidants, flavonoids, vitamins, amino acids, botanical extracts, UV filtering agents, peptides, proteins, protein hydrolysates, and/or isolates, fillers (e.g., organic and/or inorganic fillers such as talc, calcium carbonate, silica, particular materials, etc.), emollients, composition colorants, or a mixture thereof;
   wherein all percentages by weight are based on a total weight of the composition.

The pH of the composition can vary. Nonetheless, in various embodiments, a pH less than 7 (an acidic pH) is desirable. For example, the pH can be from about 3 to about 6.5, about 3 to about 6, about 3 to about 5.5, about 3 to about 5, about 3 to about 4.5, about 3 to about 4, about 3.5 to about 6.5, about 3.5 to about 6, about 3.5 to about 5.5, about 3.5 to about 5, or about 3.5 to about 4.5.

### EXAMPLES

Implementation of the present disclosure is provided by way of the following examples. The examples serve to illustrate the technology without being limiting in nature.

### Example 1

### (Compositions)

| | | Inventive | Comparative | | | |
|---|---|---|---|---|---|---|
| | | A | B | C | D | E |
| (a) | Citric Acid | 2.5 | 2.5 | 2.5 | 2.5 | 2.5 |
| (b) | Cyclodextrin | 1.5 | 1.5 | 1.5 | 1.5 | 1.5 |
| Total (a) + (b) | | 4 | 4 | 4 | 4 | 4 |
| Weight Ratio | | 1.7:1 | 1.7:1 | 1.7:1 | 1.7:1 | 1.7:1 |
| Mole Ratio | | 10:1 | 10:1 | 10:1 | 10:1 | 10:1 |
| (c) | Glycerin | 1 | | 1 | 1 | 1 |
| (d) | Hydroxypropyl Guar | 1 | 1 | | | |
| | Hydroxypropyltirmonium | | | | | |
| | Chloride | | | | | |
| | Guar Gum | | | | 1 | |
| | Carrageenan | | | | | 1 |
| | Miscellaneous * | ≤5 | ≤5 | ≤5 | ≤5 | ≤5 |
| (e) | Water | 94 | 95 | 95 | 94 | 94 |

| | | | | | |
|---|---|---|---|---|---|
| pH | 3-4 | 3-4 | 3-4 | 3-4 | 3-4 |

* Fillers, pH adjusters, preservatives, botanical extract, fragrances, buffers, stabilizers, vitamins, and/or emollients, etc., for example, henoxyethanol, sodium hydroxide, and acetic acid.

### Example 2

### (Curl Performance Without Heat)

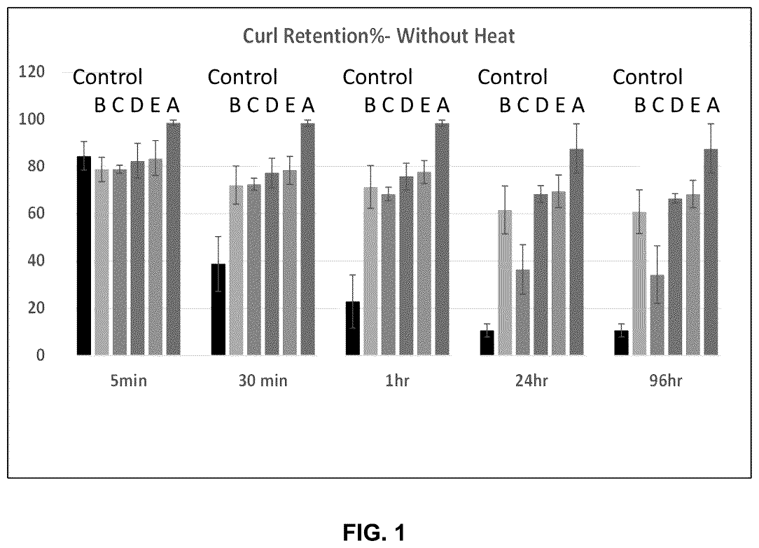

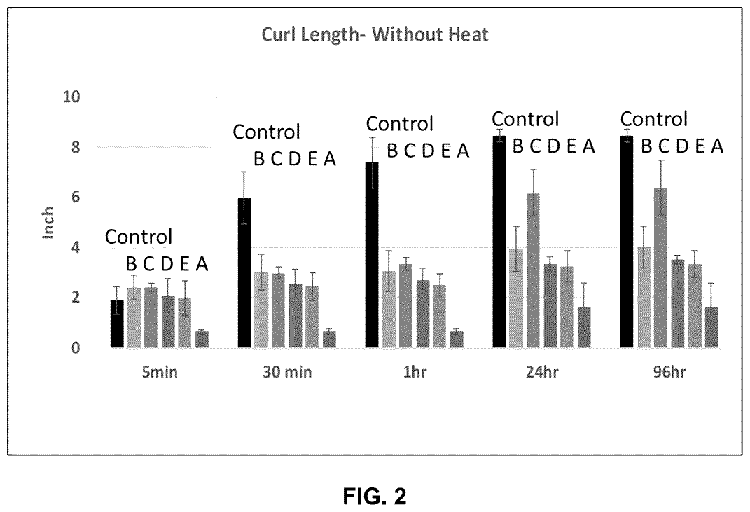

Caucasian straight hair swatches were obtained and cleansed with a standard shampoo. After rinsing the shampoo from the hair swatches, 0.15 grams (per gram of hair) of inventive composition A or comparative composition B-E were applied to the wet or damp hair swatches and massaged into the hair for about 1 minute. The hair of each hair swatch was then wrapped onto a roller. As a control, a hair swatch was cleansed with the standard shampoo and wrapped onto a roller. The hair was allowed to dry overnight on the rollers. The next day, the hair swatches were removed from the rollers and placed into a humidity chamber at 23° C. and 80% relative humidity and assessed for curl retention and curl length at 5 minutes, 30 minutes, 1 hour, 24 hours, and 96 hours. The results for curl retention are shown in FIG. 1 and the results for curl length are shown in FIG. 2. The data show that absent treatment with inventive composition A, the shape of the hair (i.e., the curls) is not maintained. Hair treated with the inventive composition showed a statistically significant improvement for both curl retention and curl length (p<0.001), as determined by Sigma® Plot 12.0 software using a one-way Anova method for all time periods compared to the control and all comparative compositions.

Curl Retention
Statistically Significant (p < 0.001)
Sigma ® Plot 12.0 software using a one-way Anova method

| | 5 min. | 30 min. | 1 hour | 24 hours | 96 hours |
|---|---|---|---|---|---|
| Control | Yes | Yes | Yes | Yes | Yes |
| Comparative B | Yes | Yes | Yes | Yes | Yes |
| Comparative C | Yes | Yes | Yes | Yes | Yes |
| Comparative D | Yes | Yes | Yes | Yes | Yes |
| Comparative E | Yes | Yes | Yes | Yes | Yes |

Curl Length
Statistically Significant (p < 0.001)
Sigma ® Plot 12.0 software using a one-way Anova method

| | 5 min. | 30 min. | 1 hour | 24 hours | 96 hours |
|---|---|---|---|---|---|
| Control | Yes | Yes | Yes | Yes | Yes |
| Comparative B | Yes | Yes | Yes | Yes | Yes |
| Comparative C | Yes | Yes | Yes | Yes | Yes |
| Comparative D | Yes | Yes | Yes | Yes | Yes |
| Comparative E | Yes | Yes | Yes | Yes | Yes |

### Example 3

### (Curl Performance With Heat)

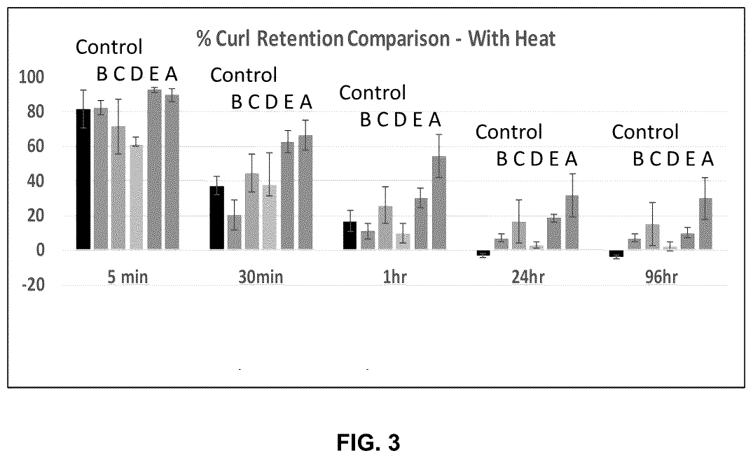

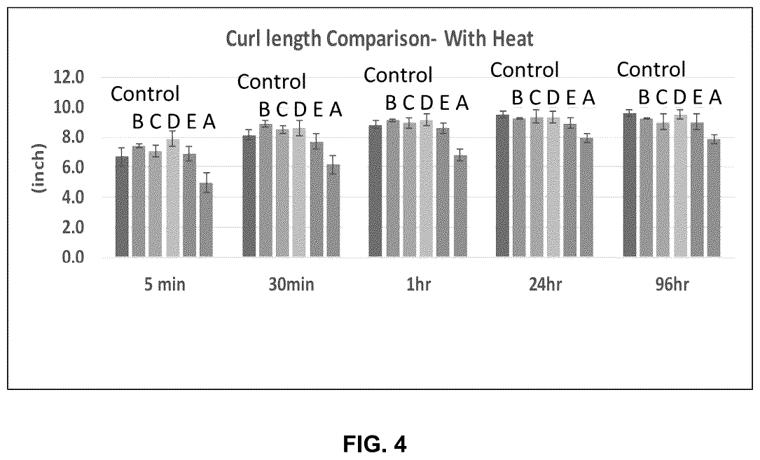

Caucasian straight hair swatches were obtained and cleansed with a standard shampoo. After rinsing the shampoo from the hair swatches, 0.15 grams (per gram of hair) of inventive composition A or comparative composition B-E were applied to the wet or damp hair swatches and massaged into the hair for about 1 minute. As a control, a hair swatch was cleansed with the standard shampoo. The hair swatches were then blow dried for about 2 minutes, until dry. A curling iron was used to curl the dry hair swatches. The hair was wrapped around the curling iron set at a temperature of 180° C. for about 20 seconds. After curling, the hair swatches were placed into a humidity chamber at 23° C. and 80% relative humidity and assessed for curl retention and curl length at 5 minutes, 30 minutes, 1 hour, 24 hours, and 96 hours. The results for curl retention are shown in FIG. 3 and the results for curl length are shown in FIG. 4. The data show that absent treatment with inventive composition A, the shape of the hair (i.e., the curls) is not maintained. Hair treated with the inventive composition showed a statistically significant improvement for both curl retention and curl length (p<0.001) at different time points, as determined by Sigma® Plot 12.0 software using a one-way Anova method compared to the control and all comparative compositions.

Curl Retention
Statistically Significant (p < 0.001)
Sigma ® Plot 12.0 software using a one-way Anova method

| | 5 min. | 30 min. | 1 hour | 24 hours | 96 hours |
|---|---|---|---|---|---|
| Control | No | No | Yes | Yes | Yes |
| Comparative B | No | Yes | Yes | No | No |
| Comparative C | No | No | Yes | No | No |
| Comparative D | Yes | No | Yes | No | No |
| Comparative E | No | No | Yes | No | No |

Curl Length
Statistically Significant (p < 0.001)
Sigma ® Plot 12.0 software using a one-way Anova method

| | 5 min. | 30 min. | 1 hour | 24 hours | 96 hours |
|---|---|---|---|---|---|
| Control | No | Yes | Yes | Yes | Yes |
| Comparative B | Yes | Yes | Yes | Yes | Yes |
| Comparative C | Yes | Yes | Yes | No | No |
| Comparative D | Yes | Yes | Yes | Yes | Yes |
| Comparative E | No | Yes | Yes | No | No |

### Example 3

### (Wash Study)

Caucasian straight hair swatches (n=6) were obtained and cleansed with a standard shampoo. After rinsing the shampoo from the hair swatches, 0.15 grams (per gram of hair) of inventive composition A was applied to the wet or damp hair swatches and massaged into the hair for about 1 minute. The hair of each hair swatch was then wrapped onto a roller. As a control, a hair swatch was cleansed with the standard shampoo and wrapped onto a roller. The hair swatches were allowed to dry overnight on the rollers. The next day, the hair was removed from the rollers washed with a standard shampoo and placed into a humidity chamber at 23° C. and 80% relative humidity for 24 hours. After 24 hours in the humidity chamber, the hair swatches were tested using a Rumba device by Bass Nova Vision to determine frizz volume, curl definition, and curliness. The Rumba device has been specifically designed to measure the orientation of hair fibers, and compute straightness and alignment coefficients. It is commonly used in the cosmetic industry. It is useful for all types of hair. The results are reported in the table below.

| | Inventive | Control |
|---|---|---|
| Frizz Volume | *70.5 cm² | 101.9 cm² |
| Curl Definition | *45.2 avg. | 32.1 (1/°) |
| | alignment coef. (1/°) | |
| Curliness | *16.2° | 13.5° |

*Statistically significant (p < 0.05)

The data show that the hair swatches treated with inventive composition A were significantly less frizzy, had better curl definition, and retained more curliness. The results for all three measurements were statistically significant (p<0.05), as determined by Sigma® Plot 12.0 software using a one-way Anova analysis.

The foregoing description illustrates and describes the disclosure. Additionally, the disclosure shows and describes only the preferred embodiments. However, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

As used herein, the terms “comprising,” “having,” and “including” are used in their open, non-limiting sense.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting. Appropriate counterions for the components described herein are known in the art.

The expression “one or more” means “at least one” and thus includes individual components as well as mixtures/combinations.

The term “plurality” means “more than one” or “two or more.”

Other than in the operating examples, or where otherwise indicated, all numbers expressing quantities of ingredients and/or reaction conditions may be modified in all instances by the term “about,” meaning within +/−5% of the indicated number.

All percentages, parts and ratios herein are based upon the total weight of the compositions of the present invention, unless otherwise indicated.

Some of the various categories of components identified may overlap. In such cases where overlap may exist and the composition includes both components (or the composition includes more than two components that overlap), an overlapping compound does not represent more than one component. For example, certain compounds may be considered both a nonionic surfactant or emulsifier and a fatty compound. If a particular composition includes both a nonionic surfactant or emulsifier and a fatty compound, a single compound will serve as only the nonionic surfactant or emulsifier or only as the fatty compound (the single compound does not simultaneously serve as both the nonionic surfactant or emulsifier and the fatty component).

A “rinse-off” product refers to a composition that is rinsed and/or washed from the hair with water either after or during the application of the composition onto the hair, and before drying and/or styling the hair. At least a portion of the composition is removed from the hair during the rinsing and/or washing.

A “leave-on” product refers to a composition that is not rinsed and/or washed from the hair after or during application of the composition onto the hair. The composition remains on the hair during drying and/or throughout styling.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The composition of the instant case optionally include one or more surfactants and/or emulsifiers, for example, one or more nonionic, anionic, cationic, and/or amphoteric/zwitterionic surfactants. The term “surfactants” and “emulsifiers” include salts of the surfactants and emulsifiers even if not explicitly stated. In other words, whenever the disclosure refers to a surfactant or emulsifier, it is intended that salts are also encompassed to the extent such salts exist, even though the specification may not specifically refer to a salt (or may not refer to a salt in every instance throughout the disclosure), for example, by using language such as “a salt thereof” or “salts thereof.” Sodium and potassium are common cations that form salts with surfactants and emulsifiers. However, additional cations such as ammonium ions, or alkanolammonium ions such as monoethanolammonium or triethanolammonium ions, may also form salts of surfactants.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, or none of the specified material.

All components that are positively set forth in the instant disclosure may be negatively excluded from the claims, e.g., a claimed composition may be “free,” “essentially free” (or “substantially free”) of one or more components that are positively set forth in the instant disclosure.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A method for shaping hair comprising:
(i) applying a hair shaping composition to the hair;
(ii) physically manipulating the hair upon which the hair shaping composition is applied into a desired shape; and
(iii) without removing the shaping composition from the hair, drying the hair;
wherein the hair shaping composition comprises:
(a) about 1 to about 8 wt. % of citric acid, a salt thereof, or a combination thereof;
(b) about 0.5 to about 5 wt. % of one or more cyclodextrins, derivatives thereof, or a combination thereof;
wherein a combined total amount of (a) and (b) is about 2 to about 12 wt. %;
(c) about 0.1 to about 5 wt. % of one or more water soluble solvents;
(d) about 0.1 to about 8 wt. % of one or more cationic guar derivatives; and
(e) about 70 to about 96 wt. % of water;
wherein all percentages by weight are based on a total weight of the composition.

2. The method of claim 1, wherein the hair shaping composition has a pH of about 3 to about 6.

3. The method of claim 1, wherein (a) and (b) are in a weight ratio of about 4:1 to about 1:1 ((a):(b)) and/or a molar ratio of about 20:1 to about 5:1 ((a):(b)).

4. The method of claim 1, wherein (a) and (b) are individually combined with each other to form a mixture in which (b) is dissolved (a); and the mixture is subsequently combined with additional components of the composition.

5. The method of claim 1, wherein the one or more cyclodextrins, derivatives thereof, or a mixture thereof, are selected from α-cyclodextrin, β-cyclodextrin, γ-cyclodextrin, methyl-α-cyclodextrin, methyl-β-cyclodextrin, methyl-γ-cyclodextrin, or mixtures thereof.

6. The method of claim 1, wherein the one or more cyclodextrins, derivatives thereof, or a mixture thereof are selected from β-cyclodextrin, derivatives thereof, or mixtures thereof.

7. The method of claim 1, wherein the composition is free from film-forming polymers.

8. The method of claim 1, wherein the hair shaping composition further comprises:
(f) about 0.1 to about 10 wt. % of one or more miscellaneous ingredients.

9. The method of claim 8, wherein the one or more miscellaneous ingredients are selected from preservatives, fragrances, pH adjusters, salts, chelating agents, buffers, antioxidants, flavonoids, vitamins, amino acids, botanical extracts, UV filtering agents, peptides, proteins, protein hydrolysates, and/or isolates, fillers (e.g., organic and/or inorganic fillers such as talc, calcium carbonate, silica, particular materials, etc.), emollients, composition colorants, or mixtures thereof.

10. The method of claim 1, wherein the hair shaping composition comprises:
(a) about 1 to about 5 wt. % of citric acid, salt thereof, or combination thereof;
(b) about 1 to about 5 wt. % of one or more cyclodextrins, derivatives thereof, or a combination thereof;
wherein a combined total amount of (a) and (b) is about 2 to about 8 wt. %;
(c) about 0.5 to about 4 wt. % of one or more water soluble solvents;
(d) about 0.5 to about 5 wt. % of hydroxypropyl guar hydroxypropyltrimonium chloride;
(e) about 70 to about 96 wt. % of water;
(f) optionally, about 0.1 to about 10 wt. % of one or more miscellaneous ingredients;
wherein the composition has a pH of about 3 to about 5, and all percentages by weight are based on a total weight of the composition.

11. The method of claim 10, wherein (a) and (b) are in a weight ratio of about 4:1 to about 1:1 ((a):(b)) and/or a molar ratio of about 20:1 to about 5:1.

12. The method of claim 11, wherein (a) and (b) are individually combined with each other to form a mixture in which the cyclodextrin is dissolved in the citric acid; and the mixture is subsequently combined with additional components of the composition.

13. The method of claim 11, wherein the one or more cyclodextrins, derivatives thereof, or a mixture thereof, are selected from β-cyclodextrin, derivatives thereof, or mixtures thereof.

14. The method of claim 1, wherein the hair is physically manipulated into the shape of curls.

15. The method of claim 14, wherein the hair is wrapped around curlers and dried while wrapped around the curlers.

16. The method of claim 1, further comprising heating the hair upon which the hair shaping composition is applied, to a temperature of about 140° C. to about 220° C., wherein the heating is optionally preceded by a pause time of from 1 minute to about 1 hour.

17. The method of claim 1, wherein drying the hair comprises allowing the hair to dry naturally under ambient conditions.

18. The method of claim 1, wherein drying the hair comprises drying the hair with a blow dryer.

19. The method of claim 1, further comprising curling the hair with a curling iron at a temperature of about 140° C. to about 220° C., wherein the curling with the curling iron is optionally preceded by a pause time of from 1 minute to about 1 hour.

20. The method of claim 1 comprising drying the hair with a blow dryer and subsequently shaping the hair with a hot iron at a temperature of about 140° C. to about 220° C.
